(19) United States
(12) Patent Application Publication
(10) Pub. No.: US 2026/0186090 A1
(43) Pub. Date: Jul. 2, 2026

(54) METHOD FOR DETERMINING SCANNING PROTOCOL IN MEDICAL IMAGING AND MEDICAL IMAGING SYSTEM

(71) Applicant: GE Precision Healthcare LLC, Waukesha, WI (US)

(72) Inventors: Ting Ye, Beijing (CN); Hao Yang, Beijing (CN); Quan Zhang, Beijing (CN); Fan Li, Beijing (CN); Qiang Xu, Beijing (CN)

(21) Appl. No.: 19/427,566

(22) Filed: Dec. 19, 2025

(30) Foreign Application Priority Data
Dec. 26, 2024 (CN) 202411942087.4

(51) Int. Cl. G01R 33/54; A61B 5/055; G06T 7/73

(52) U.S. Cl. CPC G01R 33/543; G06T 7/74; A61B 5/055; G06T 2207/10028; G06T 2207/30004; G06T 2207/30196

(57) ABSTRACT

A method for determining a scanning protocol in medical imaging and a medical imaging system are provided. The method includes determining a scanning protocol based on at least two of the following: occlusion information of key points on an examined subject, connection status information between a coil covering the surface of the examined subject and a medical scanning device, and orientation information of the examined subject. The scanning protocol is used by the medical scanning device to scan the examined subject, and the method further includes notifying the scanning protocol. This approach eliminates the need to manually load the scanning protocol, simplifying the scanning process and improving scanning efficiency.

### CROSS REFERENCE TO RELATED APPLICATIONS

The present application claims priority and benefit of Chinese Patent Application No. 202411942087.4 filed on Dec. 26, 2024, which is incorporated herein by reference in its entirety.

### TECHNICAL FIELD

Embodiments of the present application relate to the technical field of medical devices, and in particular relate to a method for determining a scanning protocol in medical imaging and a medical imaging system.

### BACKGROUND

In a scenario in which a medical imaging system is used to scan and image a subject under detection, a scanning protocol needs to be determined for the subject under detection, for example, locations of key points on the subject under detection are determined, anatomical structures to be scanned are determined based on the key points, and a scanning sequence is determined for multiple anatomical structures.

In a medical imaging system having a camera, a two-dimensional image and a depth image of a subject under detection may be captured by the camera, and the two-dimensional image and the depth image captured by the camera are recognized by using a deep learning (DL) model to determine anatomical key points of the subject under detection. A user selects one of the anatomical key points from anatomical information in a 3-plane localizer protocol as a scanning landmark, thereby setting the 3-plane localizer protocol. The user needs to manually load the 3-plane localizer protocol outside a scan room.

For example, when the head needs to be scanned, a 3-plane localizer protocol applicable to the head is manually loaded. For another example, when the chest needs to be scanned, a 3-plane localizer protocol applicable to the chest is manually loaded.

It should be noted that the above introduction of the background is only for the convenience of clearly and completely describing the technical solutions of the present application, and for the convenience of understanding for those skilled in the art.

### SUMMARY OF THE INVENTION

The inventor of the present application has found that, in the prior art, a user usually needs to manually load a 3-plane localizer protocol containing anatomical information outside a scan room, resulting in a complex scanning process and low scanning work efficiency.

In order to solve the above technical problem or at least similar technical problems, embodiments of the present application provide a method for determining a scanning protocol in medical imaging and a medical imaging system. The method can automatically determine a scanning protocol for an examined subject (for example, the scanning protocol comprises a 3-plane localizer protocol). Therefore, a user need not manually load the scanning protocol outside a scan room, thereby simplifying the scanning process and improving scanning efficiency.

According to one aspect of the embodiments of the present application a method for determining a scanning protocol in medical imaging is provided. The method includes determining a scanning protocol based on at least two of the following: occlusion information of key points on an examined subject, connection status information between a coil covering the surface of the examined subject and a medical scanning device, and orientation information of the examined subject. The scanning protocol is used by the medical scanning device to scan the examined subject, and the method further includes notifying the scanning protocol.

According to another aspect of the embodiments of the present application, a medical imaging system is provided. The system comprises: a controller, configured to execute the above-mentioned method for determining a scanning protocol.

One of the beneficial effects of the embodiments of the present application is that: Based on at least two of the following: occlusion information of key points on an examined subject, connection status information of a coil covering the surface of the examined subject, and orientation information of the examined subject, the method can automatically determine a scanning protocol for the examined subject (for example, the scanning protocol comprises a 3-plane localizer protocol), and a user need not manually load the scanning protocol outside a scan room, thereby simplifying the scanning process and improving scanning efficiency.

With reference to the following description and drawings, specific implementations of the embodiments of the present application are disclosed in detail, and the way in which the principles of the embodiments of the present application can be employed are illustrated. It should be understood that the implementations of the present application are not limited in scope thereby. Within the scope of the spirit and clauses of the appended claims, the implementations of the present application comprise many changes, modifications, and equivalents.

### BRIEF DESCRIPTION OF THE DRAWINGS

The included drawings are used to provide further understanding of the embodiments of the present application, which constitute a part of the description and are used to illustrate the implementations of the present application and explain the principles of the present application together with textual description. Evidently, the drawings in the following description are merely some embodiments of the present application, and those of ordinary skill in the art may obtain other implementations according to the drawings without involving inventive effort. In the drawings:

FIG. 1 is a schematic diagram of a magnetic resonance imaging system according to an embodiment of the present application;

FIG. 2 is a schematic diagram of a method for determining a scanning protocol in medical imaging according to an embodiment of the present application;

FIG. 3 is a schematic diagram of key points on an examined subject according to an embodiment of the present application;

FIG. 4 is a schematic diagram of a method for acquiring orientation information of an examined subject;

FIG. 5 is a schematic diagram of a method for acquiring occlusion information of key points on an examined subject;

FIG. 6 is a schematic diagram of a process for determining a scanning protocol according to an embodiment of the present application;

FIG. 7 is another schematic diagram of a process for determining a scanning protocol according to an embodiment of the present application;

FIG. 8 is a schematic diagram of a process for determining two scanning protocols according to an embodiment of the present application;

FIG. 9 is another schematic diagram of a process for determining two scanning protocols according to an embodiment of the present application;

FIG. 10 is a schematic diagram of a process for determining two or more scanning protocols according to an embodiment of the present application;

FIG. 11 is another schematic diagram of a process for determining two or more scanning protocols according to an embodiment of the present application;

FIG. 12 is still another schematic diagram of a process for determining two or more scanning protocols according to an embodiment of the present application;

FIG. 13 is still another schematic diagram of a process for determining two or more scanning protocols according to an embodiment of the present application;

FIG. 14 is a schematic diagram of an apparatus for determining a scanning protocol according to an embodiment of the present application; and

FIG. 15 is a schematic diagram of a medical imaging system according to an embodiment of the present application.

### DETAILED DESCRIPTION

The aforementioned and other features of the embodiments of the present application will become apparent from the following description with reference to the drawings. In the description and drawings, specific implementations of the present application are disclosed in detail, and part of the implementations in which the principles of the embodiments of the present application may be employed are indicated. It should be understood that the present application is not limited to the described implementations. On the contrary, the embodiments of the present application include all modifications, variations, and equivalents which fall within the scope of the appended claims.

In the embodiments of the present application, the terms “first”, “second”, etc., are used to distinguish different elements with respect to naming, but do not represent a spatial arrangement or temporal order, etc., of these elements, and these elements should not be limited by these terms. The term “and/or” includes any and all combinations of one or more associated listed terms. The terms “comprise”, “include”, “have”, etc., refer to the presence of described features, elements, components, or assemblies, but do not exclude the presence or addition of one or more other features, elements, components, or assemblies.

In the embodiments of the present application, the singular forms “a” and “the” include the plural forms, and should be broadly construed as “a type of” or “a class of” rather than being limited to the meaning of “one”. Furthermore, the term “the” should be construed as including both the singular and plural forms, unless otherwise specified in the context. In addition, the term “according to” should be construed as “at least in part according to . . . ” and the term “based on” should be construed as “at least in part based on . . . ”, unless otherwise specified in the context.

In the embodiments of the present application, the term “key point” may be equivalently replaced with “key coordinate point”, “landmark”, “landmark point”, or the like. The term “subject” may be equivalently replaced with “subject under detection”, “detected subject”, “subject under examination” “examined subject”, “scanned subject”, “subject to be scanned”, “patient”, “subject of study”, or the like, which may be a human being or an animal, or may be other subjects.

In the embodiments of the present application, the term “include/comprise” when used herein refers to the presence of features, integrated components, steps, or assemblies, but does not preclude the presence or addition of one or more other features, integrated components, steps, or assemblies.

The features described and/or illustrated for one implementation may be used in one or more other implementations in the same or similar way, be combined with features in other implementations, or replace features in other implementations.

In the embodiments of the present application, a method for determining an orientation of a subject or an apparatus for determining an orientation of a subject may be applicable to various medical imaging scenarios, including, but not limited to, magnetic resonance imaging (MRI), computed tomography (CT), ultrasound imaging, positron emission computed tomography (PET), single photon emission computed tomography (SPECT), PET/CT, PET/MR, or any other suitable medical imaging scenarios.

In the embodiments of the present application, the method, apparatus, and system of the present application are exemplarily described by taking an MRI scenario as an example. It should be understood that the contents of the embodiments of the present application are also applicable to other medical imaging scenarios.

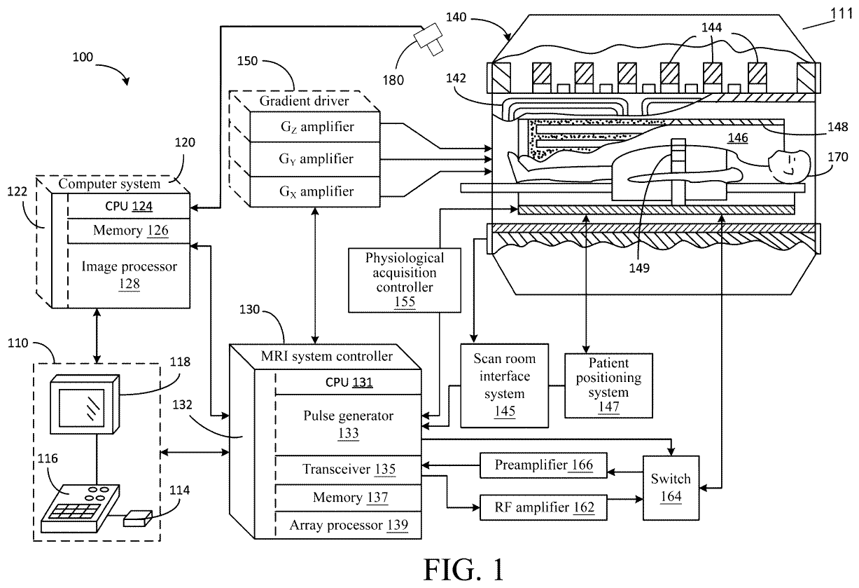

For ease of understanding, FIG. 1 is a schematic diagram of a magnetic resonance imaging (MRI) system 100 according to an embodiment of the present application.

The MRI system 100 includes a scanning unit 111. The scanning unit 111 is used to perform a magnetic resonance scan of a subject (e.g., a human body) 170 to generate image data of a region of interest of the subject 170, wherein the region of interest may be a pre-determined anatomical site or anatomical tissue.

The operation of the MRI system 100 is controlled by an operator workstation 110 that includes an input device 114, a control panel 116, and a display 118. The input device 114 may be a joystick, a keyboard, a mouse, a trackball, a touch-activated screen, voice control, or any similar or equivalent input device. The control panel 116 may include a keyboard, a touch-activated screen, voice control, a button, a slider, or any similar or equivalent control device. The operator workstation 110 is coupled to and communicates with a computer system 120 that enables an operator to control the generation and display of images on the display 118. The computer system 120 includes various components that communicate with one another via an electrical and/or data connection module 122. The connection module 122 may employ a direct wired connection, a fiber optic connection, a wireless communication link, etc. The computer system 120 may include a central processing unit (CPU) 124, a memory 126, and an image processor 128. In some embodiments, the image processor 128 may be replaced by medical imaging functions implemented in the CPU 124. The computer system 120 may be connected to an archive media device, a persistent or backup memory, or a network. The computer system 120 may be coupled to and communicates with a separate MRI system controller 130.

The MRI system controller 130 includes a set of components that communicate with one another via an electrical and/or data connection module 132. The connection module 132 may employ a direct wired connection, a fiber optic connection, a wireless communication link, etc. The MRI system controller 130 may include a CPU 131, a sequence pulse generator (also known as a pulse generator) 133 communicating with the operator workstation 110, a transceiver (also known as an RF transceiver) 135, a memory 137, and an array processor 139.

In some embodiments, the sequence pulse generator 133 may be integrated into a resonance assembly 140 of the scanning unit 111 of the MRI system 100. The MRI system controller 130 may receive a command from the operator workstation 110, and is coupled to the scanning unit 111 to indicate an MRI scanning sequence to be executed during an MRI scan, so as to be used to control the scanning unit 111 to execute the flow of the aforementioned magnetic resonance scan. The MRI system controller 130 is further coupled to a gradient driver system (also known as gradient driver) 150 and communicates therewith, and the gradient driver system is coupled to a gradient coil assembly 142 to generate a magnetic field gradient during an MRI scan.

The sequence pulse generator 133 may further receive data from a physiological acquisition controller 155 that receives signals from a plurality of different sensors (e.g., electrocardiogram (ECG) signals from electrodes attached to a patient, etc.), the sensors being connected to a subject or patient 170 undergoing an MRI scan. The sequence pulse generator 133 is coupled to and communicates with a scan room interface system 145 that receives signals from various sensors associated with the state of the resonance assembly 140. The scan room interface system 145 is further coupled to and communicates with a patient positioning system 147 that sends and receives signals to control a patient table to move to a desired position for performing an MRI scan.

The MRI system controller 130 provides gradient waveforms to the gradient driver system 150, and the gradient driver system includes Gₓ (x direction), G_{y} (y direction), and G_{z} (z direction) amplifiers, etc. Each of the Gₓ, G_{y}, and G_{z} gradient amplifiers excites a corresponding gradient coil in the gradient coil assembly 142, so as to generate a magnetic field gradient used to spatially encode an MR signal during an MRI scan. The gradient coil assembly 142 is disposed within the resonance assembly 140, and the resonance assembly further includes a superconducting magnet having a superconducting coil 144 that, in operation, provides a static uniform longitudinal magnetic field B₀ throughout a cylindrical imaging volume 146. The resonance assembly 140 further includes an RF body coil 148, which, in operation, provides a transverse magnetic field B₁, the transverse magnetic field B₁ being substantially perpendicular to B₀ throughout the entire cylindrical imaging volume 146. The resonance assembly 140 may further include an RF surface coil 149 for imaging different anatomical structures of a patient undergoing an MRI scan. The RF body coil 148 and the RF surface coil 149 may be configured to operate in a transmit and receive mode, a transmit mode, or a receive mode.

The x direction may also be referred to as a frequency encoding direction or a kₓ direction in the k-space, the y direction may be referred to as a phase encoding direction or a k_{y} direction in the k-space, and the z direction may be referred to as a layer surface selection (layer selection) direction. Gₓ can be used for frequency encoding or signal readout, and is generally referred to as a frequency encoding gradient or a readout gradient. G_{y} can be used for phase encoding, and is generally referred to as a phase encoding gradient. G_{z} can be used for slice (layer) position selection to obtain k-space data. It should be noted that a layer selection direction, a phase encoding direction, and a frequency encoding direction may be modified according to actual requirements.

The subject or patient 170 of the MRI scan may be positioned within the cylindrical imaging volume 146 of the resonance assembly 140. The transceiver 135 in the MRI system controller 130 generates RF excitation pulses amplified by an RF amplifier 162, and provides the same to the RF body coil 148 by means of a transmit/receive switch (also known as T/R switch or switch) 164.

As described above, the RF body coil 148 and the RF surface coil 149 may be used to transmit RF excitation pulses and/or receive obtained MR signals from a patient undergoing an MRI scan. The MR signals emitted by excited nuclei in the patient of the MRI scan may be sensed and received by the RF body coil 148 or the RF surface coil 149 and sent back to a preamplifier 166 by means of the T/R switch 164. The T/R switch 164 may be controlled by a signal from the sequence pulse generator 133 to electrically connect the RF amplifier 162 to the RF body coil 148 in the transmit mode and to connect the preamplifier 166 to the RF body coil 148 in the receive mode. The T/R switch 164 may further enable the RF surface coil 149 to be used in the transmit mode or the receive mode.

In some embodiments, the MR signals sensed and received by the RF body coil 148 or the RF surface coil 149 and amplified by the preamplifier 166 are stored in the memory 137 for post-processing as a raw k-space data array. A reconstructed magnetic resonance image may be obtained by transforming/processing the stored raw k-space data.

In some embodiments, the MR signals sensed and received by the RF body coil 148 or the RF surface coil 149 and amplified by the preamplifier 166 are demodulated, filtered, and digitized in a receiving portion of the transceiver 135, and transmitted to the memory 137 in the MRI system controller 130. For each image to be reconstructed, the data is rearranged into separate k-space data arrays, each of these separate k-space data arrays is input into the array processor 139, and the array processor is operated to transform the data into an array of image data by Fourier transform.

The array processor 139 uses transform methods, most commonly Fourier transform, to create images from received MR signals. These images are transmitted to the computer system 120 and stored in the memory 126. In response to commands received from the operator workstation 110, the image data may be stored in a long-term memory, or may be further processed by the image processor 128 and transmitted to the operator workstation 110 for presentation on the display 118.

In various embodiments, components of the computer system 120 and the MRI system controller 130 may be implemented on the same computer system or on a plurality of computer systems. It should be understood that the MRI system 100 shown in FIG. 1 is intended for illustration. Suitable MRI systems may include more, fewer, and/or different components.

The MRI system controller 130 and the image processor 128 may separately or collectively include a computer processor and a storage medium. The storage medium records a predetermined data processing program to be executed by the computer processor. For example, the storage medium may store a program used to implement scanning processing (such as a scan flow and an imaging sequence), image reconstruction, medical imaging, etc. For example, the storage medium may store a computer program for determining an orientation of a subject according to the embodiments of the present invention. The described storage medium may include, for example, a ROM, a floppy disk, a hard disk, an optical disk, a magneto-optical disk, a CD-ROM, or a non-volatile memory card.

A camera 180 may take photos of the subject under detection 170.

The inventor found that in some medical scenarios, when a medical imaging system is used to scan an examined subject, a user needs to manually load a scanning protocol such as a 3-plane localizer protocol outside a scan room, resulting in a complex scanning process and low scanning efficiency.

To solve at least one of the above problems, the embodiments of the present application provide a method and an apparatus for determining a scanning protocol in medical imaging, and a medical imaging system.

Description is made below in conjunction with the embodiments.

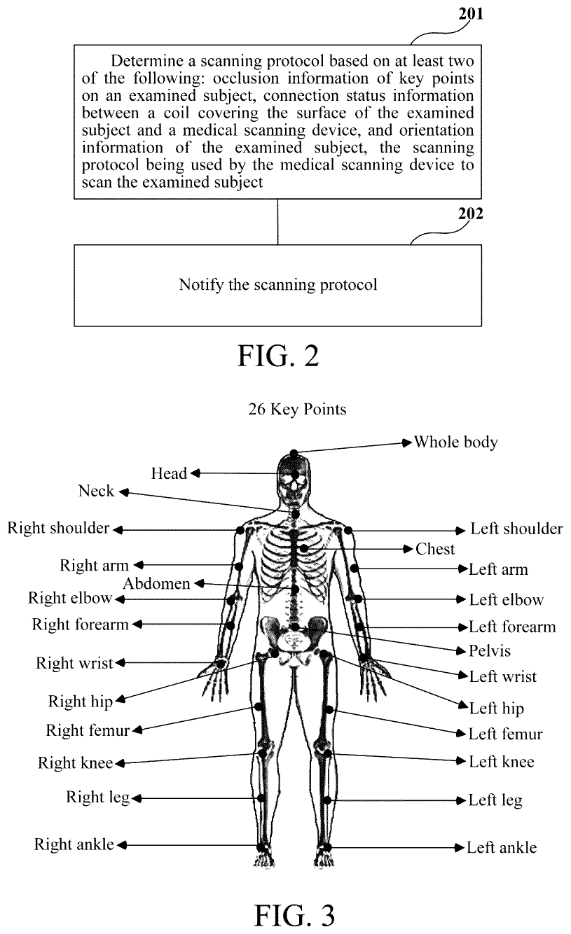

The embodiments of the present application provide a method for determining a scanning protocol in medical imaging. FIG. 2 is a schematic diagram of the method for determining a scanning protocol in medical imaging according to an embodiment of the present application.

As shown in FIG. 2, the method includes, at step 201, determining a scanning protocol based on at least two of the following: occlusion information of key points on an examined subject, connection status information between a coil covering the surface of the examined subject and a medical scanning device, and orientation information of the examined subject. The scanning protocol is used by the medical scanning device to scan the examined subject. At step 202, the method includes notifying the scanning protocol.

In the present application, the examined subject may be a human body, and the key points on the examined subject may be anatomical key points on a human body.

According to the above embodiment, the scanning protocol is determined based on at least two of the following: occlusion information of key points on an examined subject, connection status information of a coil covering the surface of the examined subject, and orientation information of the examined subject. Therefore, the scanning protocol for the examined subject can be automatically determined, and a user need not manually load the scanning protocol outside a scan room, thereby simplifying the scanning process and improving scanning efficiency.

The scanning protocol includes, for example, a 3-plane localizer protocol. For example, before a formal scan is performed on a detected subject, a rapid 3-plane localizer scan is performed on the detected subject to more accurately determine a scan region and range during the formal scan, and a scan protocol used by the 3-plane localizer scan is the 3-plane localizer protocol.

In some embodiments, at step 201, the occlusion information of the key points on the examined subject and the orientation information of the examined subject may be determined based on an image of the examined subject captured. The image may be from a camera disposed in a medical imaging system, for example, the camera 180 shown in FIG. 1. Each frame of the image captured by the camera 180 may include at least one of color channel information and depth information. The color channel information may be stored in the form of a color image, and the depth information may be stored in the form of a depth image.

Each color image may include a plurality of pixels, and a value of each pixel may include a value of each color component. The color components may include a red (R) component, a green (G) component, a blue (B) component, and the like.

Each depth image may include a plurality of pixels, and a value of each pixel may include a depth value (D) of the pixel. The depth value of a pixel may represent a distance to the camera from a point on a subject (e.g., a subject to be captured within a capturing range of the camera 180) that corresponds to the pixel.

In some embodiments, the camera may acquire a depth image sequence and a color image sequence during a period of time. The color image sequence may include a plurality of color images, and the depth image sequence may include a plurality of depth images. Each color image corresponds to a different time instance, and each depth image corresponds to a different time instance. The color images in the color image sequence may be in a one-to-one correspondence to the depth images in the depth image sequence. For example, the color images in the color image sequence may be aligned in time with the depth images in the depth image sequence. The present application is not limited thereto. The color images in the color image sequence may also be in other correspondence relationships with the depth images in the depth image sequence.

In the above embodiments, the camera may be placed near (e.g., on an upper side of) the scanning unit 111 shown in FIG. 1, to capture the subject under detection 170 from an overhead perspective and obtain an image. For example, it may be placed at the position of the camera 180 as shown in FIG. 1. In addition, the present application is not limited thereto, and the camera may also be disposed at other positions.

In some embodiments, at step 201, the key points on the examined subject may be, for example, feature points that are related to a body part or anatomical structure and can be detected in the image. The types of the key points may include at least one of the following types: a head key point, a chest key point, an abdomen key point, a neck key point, a nose key point, a left shoulder key point, a right shoulder key point, a left hip key point, a right hip key point, a left eye key point, a right eye key point, a left elbow key point, a right elbow key point, a left knee key point, a right knee key point, a left ear key point, a right ear key point, a left wrist key point, a right wrist key point, a left ankle key point, a right ankle key point, and the like. The present application is not limited thereto, and the types of the key points may also include other types.

FIG. 3 is a schematic diagram of key points on an examined subject according to an embodiment of the present application. Taking FIG. 3 as an example, there may be 26 key points on the examined subject, including a head key point, a neck key point, a left shoulder key point, a right shoulder key point, a chest key point, a left arm key point, a right arm key point, a left elbow key point, a right elbow key point, a left forearm key point, a right forearm key point, a left wrist key point, a right wrist key point, an abdomen key point, a pelvis key point, a left hip key point, a right hip key point, a left femur key point, a right femur key point, a left knee key point, a right knee key point, a left leg key point, a right leg key point, a left ankle key point, a right ankle key point, and a key point representing the whole body.

One anatomical structure of the subject under examination may correspond to one key point, for example, the head may correspond to the head key point. Or, one anatomical structure may correspond to a plurality of key points, for example, the shoulder may correspond to the left shoulder key point and the right shoulder key point.

Hereinafter, acquisition of orientation information of the examined subject based on an image of the examined subject will be described.

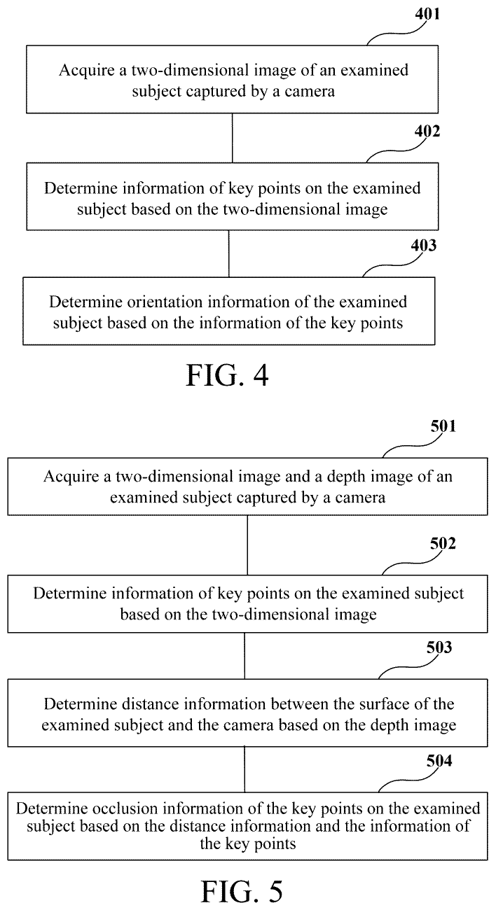

FIG. 4 is a schematic diagram of a method for acquiring orientation information of an examined subject. As shown in FIG. 4, the method includes, at step 401, acquiring a two-dimensional image of the examined subject captured by a camera. At step 402, information of key points on the examined subject is determined based on the two-dimensional image. At step 403, orientation information of the examined subject is determined based on the information of the key points.

At step 401, the two-dimensional image of the examined subject may be a color image including R, G, and B components captured by the camera 180.

In some embodiments of step 402, the two-dimensional image obtained in step 401 may be recognized by using a deep learning (DL) model to determine the information of the key points on the examined subject. In addition, the information of the key points may also be obtained in other ways. The key points on the examined subject are, for example, at least part of the 26 key points shown in FIG. 3. The information of the key points may include location information of the key points, for example, the location information of the key points may be represented by means of pixel locations in the image. The present application is not limited thereto, and the location information of the key points may also be represented in other ways.

The DL model may be a convolutional neural network (CNN). The convolutional neural network is learned by using two-dimensional images of a plurality of examined subjects as an input data set and using information of key points pre-recognized in a corresponding image as an output data set, to obtain a parameter-optimized convolutional neural network. A two-dimensional image of the currently detected subject obtained from scanning is input into the convolutional neural network that performs convolution, pooling, and other operations on the two-dimensional image, to determine information of key points on the examined subject in the two-dimensional image. The convolutional neural network may be trained online, for example, model training is also performed at a stage of two-dimensional image recognition by the convolutional neural network. The convolutional neural network may also be trained offline, for example, image recognition is performed after the convolutional neural network is trained. The present application is not limited thereto, and other suitable DL models may also be used for processing.

In some embodiments of step 403, orientation information of the examined subject may be recognized by comparing the pixel locations of the key points in the image and/or relative locations between the key points. For example, the pixel locations of the key points in the image are compared with predetermined templates, to determine to which predetermined template the pixel location is closest, and an orientation corresponding to the template is determined as an orientation of the examined subject. For another example, the relative locations between the key points are compared with several predetermined relative locations, to determine to which predetermined relative location the relative locations between the key points are closest, and orientation information corresponding to the predetermined relative location is determined as the orientation of the examined subject. For example, whether the key point is a head key point or a neck key point is determined based on a preset distance and orientation relationship between the head key point and the neck key point, to determine the orientation of the subject under examination.

The orientation information of the examined subject may include which part of the body enters a scanning space first. For example, a head-in type means that the head of the examined subject enters the scanning space first, and a feet-in type means that the feet of the examined subject enter the scanning space first. The orientation information of the examined subject may also include a direction in which the face of the examined subject is oriented, for example, the examined subject may be in a prone, supine, or left or right decubitus position.

Hereinafter, acquisition of occlusion information of key points on an examined subject based on an image of the examined subject will be described.

FIG. 5 is a schematic diagram of a method for acquiring occlusion information of key points on an examined subject. As shown in FIG. 5, the method includes, at step 501, acquiring a two-dimensional image and a depth image of the examined subject captured by a camera. At step 502, determining information of key points on the examined subject based on the two-dimensional image. At step 503, determining distance information between the surface of the examined subject and the camera based on the depth image. At step 504, determining occlusion information of the key points on the examined subject based on the distance information and the information of the key points.

In some embodiments of step 501, the two-dimensional image of the examined subject may be a color image including R, G, and B components captured by the camera 180, and the depth image of the examined subject may be a depth image including a depth value (D) captured by the camera 180.

In an embodiment of step 502, the key points on the examined subject may be at least part of the 26 key points shown in FIG. 3, and the information of the key points may be obtained based on the two-dimensional image using a deep learning (DL) model, or may be obtained in other ways. The information of the key points may include the location information of the key points, and the location information of the key points may be represented by pixel locations in the image. The present application is not limited thereto, and the location information of the key points may also be represented in other ways.

In some embodiments of step 503, the distance information between the surface of the examined subject and the camera is determined based on the depth image. For example, for a depth image sequence consisting of a plurality of depth images, when a depth value of a pixel corresponding to a part of the surface of the examined subject in the image decreases relative to a depth value of a corresponding pixel in a previous image, it indicates that a distance from the part of the surface to the camera decreases (for example, the part of the surface is occluded by a foreign matter), and when a depth value of a pixel corresponding to a part of the surface of the examined subject increases, it indicates that a distance from the part of the surface to the camera increases (for example, a foreign matter is removed from the part of the surface).

In some embodiments of step 504, the occlusion information of the key points on the examined subject is determined based on the distance information (for example, obtained by means of step 503) and the information of the key points (for example, obtained by means of step 502). Based on the location information of the key points and the distance information between the surface corresponding to the key points on the examined subject to the camera, a distance from a region on the surface of the examined subject corresponding to each key point to the camera may be determined. The distance is compared with a first threshold. When the distance from the region corresponding to the key point to the camera is less than the first threshold and the area of the region is greater than a second threshold, it is determined that the key point corresponding to the region is occluded.

The key point may be occluded by a coil (for example, the coil is the RF surface coil 149 shown in FIG. 1), or may be occluded by a blanket or other coverings. Taking the coil as an example, if the distance from the region corresponding to the key point on the surface of the examined subject to the camera is less than the first threshold, and the area of the region corresponding to the key point is greater than the second threshold, it is determined that the key point corresponding to the region is occluded by the coil. Specific values of the first threshold and the second threshold may be preset.

In the above embodiment, each key point on the examined subject is detected for whether the distance from the region on the surface of the examined subject corresponding to the key point to the camera is less than the first threshold, and whether the area of the region is greater than the second threshold, and if both conditions are satisfied, it is indicated that the surface of the examined subject corresponding to the key point is occluded. In the following description, “the surface of an examined subject corresponding to a key point being occluded” may also be simply expressed as “a key point being occluded”, and the two have the same meaning.

In the present application, for one examined subject, one or a plurality of key points may be occluded, and the plurality of key points may be occluded by the same coil or another covering, or may be occluded by a plurality of coils or other coverings, respectively.

In addition, if a depth value of a region corresponding to an occluded key point increases compared with a previous depth value, it is determined that the covering occluding the key point is removed. For example, for a region corresponding to a key point, if a depth value at time instance t is greater than a depth value at time instance t−1, it is determined that the covering occluding the key point at the time instance t−1 is removed, and thus the key point is no longer occluded at the time instance t.

In some embodiments, at step 201, the connection status information between the coil covering the surface of the examined subject (for example, the coil is the RF surface coil 149 shown in FIG. 1) and the medical scanning device may be obtained by reading relevant signals of the medical scanning device. For example, when the coil is connected to the medical scanning device, the medical scanning device generates a first signal. Therefore, if the first signal is read from the medical scanning device, it is determined that the coil is connected to the medical scanning device, and if no first signal is read from the medical scanning device, it is determined that the coil is not connected to the medical scanning device. In addition, the first signal may include type information or other recognition information of the coil, so that the first signal can also be used to indicate which type of coil (for example, a head coil) or which coil is connected to the medical scanning device.

In some examples of step 201, when it is determined that a key point on the examined subject is occluded and the coil is connected to the medical scanning device, the scanning protocol is further determined based on the orientation information of the examined subject. In addition, when it is determined that no key point on the examined subject is occluded, or the coil is not connected to the medical scanning device, the medical scanning device does not perform scanning.

Hereinafter, the method for determining the scanning protocol at step 201 will be described in conjunction with specific embodiments.

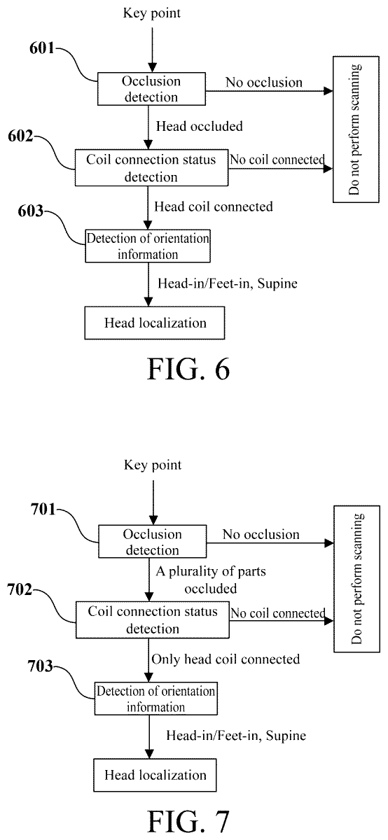

FIG. 6 is a schematic diagram of a process for determining a scanning protocol according to an embodiment of the present application, and FIG. 7 is another schematic diagram of a process for determining a scanning protocol according to an embodiment of the present application.

In the example shown in FIG. 6, the process for determining the scanning protocol includes at step 601 performing occlusion detection on key points of an examined subject to obtain occlusion information of the key points on the examined subject. For example, when it is detected that the head of the examined subject is occluded, i.e., one or more key points on the head of the examined subject are occluded, step 602 is performed. In addition, if it is detected that no key point on the examined subject is occluded (namely, there is no occlusion) at step 601, scanning is not performed.

At step 602, the process includes performing coil connection status detection. When it is detected that a coil is connected to the medical scanning device, step 603 is performed. The type of coil or which coil is connected to the medical scanning device is determined based on an indication of a first signal. For example, it is determined that a head coil is connected to the medical scanning device based on the indication of the first signal. In addition, if it is detected that no coil is connected to the medical scanning device at step 602, scanning is not performed.

At step 603, the process includes detecting orientation information of the examined subject to obtain the orientation information of the examined subject. When the orientation information of the examined subject is a head-in type or a feet-in type, and the examined subject is in a supine position, a scanning protocol suitable for head localization, e.g., a 3-plane localizer protocol for head localization, is determined.

In the example shown in FIG. 7, the process for determining the scanning protocol includes at step 701 performing occlusion detection on key points of an examined subject to obtain occlusion information of the key points on the examined subject. When it is detected that a plurality of parts of the examined subject are occluded, for example, the head, abdomen, heart, or other key parts are occluded, i.e., a plurality of key points on the above parts of the examined subject are occluded, step 703 is performed. In addition, if it is detected that no key point on the examined subject is occluded (namely, there is no occlusion) at step 702, scanning is not performed.

At step 702, the process includes performing coil connection status detection. When it is detected that a coil is connected to the medical scanning device, step 703 is performed. The type of coil or which coil is connected to the medical scanning device is determined based on an indication of a first signal. For example, it is determined that a head coil is connected to the medical scanning device based on the first signal. In addition, if it is detected that no coil is connected to the medical scanning device at step 703, scanning is not performed.

At step 703, the process includes detecting orientation information of the examined subject to obtain the orientation information of the examined subject. When the orientation information of the examined subject is a head-in type or a feet-in type, and the examined subject is in a supine position, a scanning protocol suitable for head localization, e.g., a 3-plane localizer protocol for head localization, is determined.

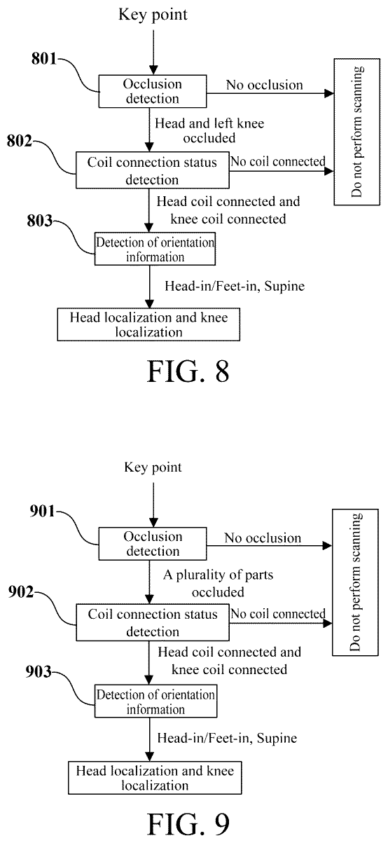

FIG. 8 is a schematic diagram of a process for determining two scanning protocols according to an embodiment of the present application, and FIG. 9 is another schematic diagram of a process for determining two scanning protocols according to an embodiment of the present application.

In the example shown in FIG. 8, the process for determining the scanning protocols includes at step 801 performing occlusion detection on key points of an examined subject to obtain occlusion information of the key points on the examined subject. For example, when it is detected that the head and left knee of the examined subject are occluded, i.e., two or more key points on the head and the left knee of the examined subject are occluded, step 802 is performed. In addition, if it is detected that no key point on the examined subject is occluded (namely, there is no occlusion) at step 801, scanning is not performed.

At step 802, the process includes performing coil connection status detection. When it is detected that a coil is connected to the medical scanning device, step 803 is performed. The type of coil or which coil is connected to the medical scanning device is determined based on an indication of a first signal. For example, it is determined that a head coil is connected to the medical scanning device and a knee coil is also connected to the medical scanning device based on the indication of the first signal. In addition, if it is detected that no coil is connected to the medical scanning device at step 803, scanning is not performed.

At step 803, the process includes detecting orientation information of the examined subject to obtain the orientation information of the examined subject. When the orientation information of the examined subject is a head-in type or a feet-in type, and the examined subject is in a supine position, scanning protocols suitable for head localization and knee localization, e.g., a 3-plane localizer protocol for head localization and a 3-plane localizer protocol for knee localization, are selected.

In the example shown in FIG. 9, the process for determining the scanning protocols includes at step 901 performing occlusion detection on key points of an examined subject. When it is detected that a plurality of parts of the examined subject are occluded, for example, the head, knee, abdomen, heart, or other key parts are occluded, i.e., a plurality of key points on the above parts of the examined subject are occluded, step 902 is performed. In addition, if it is detected that no key point on the examined subject is occluded (namely, there is no occlusion) at step 902, scanning is not performed.

At step 902, the process includes performing coil connection status detection. When it is detected that a coil is connected to the medical scanning device, step 903 is performed. The type of coil or which coil is connected to the medical scanning device is determined based on an indication of a first signal. For example, it is determined that a head coil is connected to the medical scanning device and a knee coil is connected to the medical scanning device based on the indication of the first signal. In addition, if it is detected that no coil is connected to the medical scanning device at step 902, scanning is not performed.

At step 903, the process includes detecting orientation information of the examined subject to obtain the orientation information of the examined subject. When the orientation information of the examined subject is a head-in type or a feet-in type, and the examined subject is in a supine position, scanning protocols suitable for head localization and knee localization, e.g., a 3-plane localizer protocol for head localization and a 3-plane localizer protocol for knee localization, are selected.

When two or more scanning protocols are determined, priorities of the two or more scanning protocols are determined based on at least one of: a positional relationship among a plurality of occluded key points, and a positional relationship of the plurality of occluded key points relative to the coil.

In some embodiments, the closer an occluded key point to the center of the coil, the higher the priority of the scanning protocol corresponding to the key point.

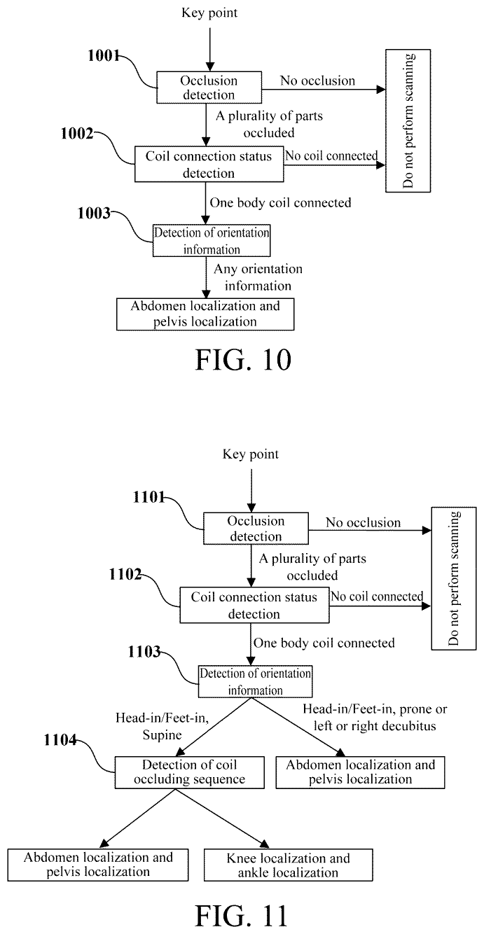

FIG. 10 is a schematic diagram of a process for determining two or more scanning protocols according to an embodiment of the present application, and FIG. 11 is another schematic diagram of a process for determining two or more scanning protocols according to an embodiment of the present application.

In the example shown in FIG. 10, the process for determining the scanning protocols includes at step 1001 performing occlusion detection on key points of an examined subject to obtain occlusion information of key points on the examined subject. When it is detected that a plurality of parts of the examined subject are occluded, for example, the heart, abdomen, pelvis, shoulders, elbows, and wrists of the examined subject are all occluded, i.e., a plurality of key points corresponding to the above parts are occluded, step 1002 is performed. In addition, if it is detected that no key point on the examined subject is occluded (namely, there is no occlusion) at step 1001, scanning is not performed.

At step 1002, the process includes performing coil connection status detection. When it is detected that a coil is connected to the medical scanning device, step 1003 is performed. The type of coil or which coil is connected to the medical scanning device is determined based on an indication of a first signal. For example, it is determined that a body coil is connected to the medical scanning device based on the indication of the first signal. In addition, if it is detected that no coil is connected to the medical scanning device at step 1002, scanning is not performed.

At step 1003, the process includes detecting orientation information of the examined subject to obtain the orientation information of the examined subject. Regardless of what type the orientation information of the examined subject is, the scanning protocol is determined to prioritize scanning a part corresponding to a key point closest to the center of the body coil. For example, a scanning protocol suitable for abdomen localization and a scanning protocol suitable for pelvis localization, e.g., a 3-plane localizer protocol for abdomen localization and a 3-plane localizer protocol for pelvis localization, are selected.

In the example shown in FIG. 11, the process for determining the scanning protocols includes at step 1101 performing occlusion detection on key points of an examined subject to obtain occlusion information of the key points on the examined subject. When it is detected that a plurality of parts of the examined subject are occluded, for example, the heart, abdomen, pelvis, shoulders, elbows, wrists, hips, knees, and ankles of the examined subject are all occluded, step 1102 is performed. In addition, if it is detected that no key point on the examined subject is occluded (namely, there is no occlusion) at step 1101, scanning is not performed.

At step 1102, the process includes performing coil connection status detection. When it is detected that a coil is connected to the medical scanning device, step 1103 is performed. The type of coil or which coil is connected to the medical scanning device is determined based on an indication of a first signal. For example, it is determined that a body coil is connected to the medical scanning device based on the indication of the first signal. In addition, if it is detected that no coil is connected to the medical scanning device at step 1102, scanning is not performed.

At step 1103, the process includes detecting orientation information of the examined subject to obtain the orientation information of the examined subject. When the orientation information is a head-in type or a feet-in type, and the examined subject is in a prone or left or right decubitus position, scanning protocols suitable for abdomen/pelvis localization, e.g., 3-plane localizer protocols for abdomen/pelvis localization, are selected. When the orientation information is a head-in type or a feet-in type, and the examined subject is in a supine position, step 1104 is performed.

At step 1104, the process includes detecting a coil occlusion sequence to obtain the sequence in which the coil occludes the examined subject. For example, when the body coil preferentially occludes the heart, abdomen, pelvis, shoulders, elbows, wrists, and other parts of the examined subject, the scanning protocol is determined to prioritize scanning a part corresponding to a key point closest to the center of a region preferentially occluded by the body coil. For example, a scanning protocol suitable for abdomen localization and a scanning protocol suitable for pelvis localization, e.g., a 3-plane localizer protocol for abdomen localization and a 3-plane localizer protocol for pelvis localization, are selected. When the body coil preferentially occludes the hips, knees, ankles, and other parts of the examined subject, the scanning protocol is determined to prioritize scanning a part corresponding to a key point closest to the center of a region preferentially occluded by the body coil. For example, a scanning protocol suitable for knee localization and a scanning protocol suitable for ankle localization, e.g., a 3-plane localizer protocol for knee localization and a 3-plane localizer protocol for ankle localization, are selected.

In the above example, the closer a key point is to the center of the coil, the higher a scanning priority corresponding to the key point. The present application is not limited thereto. It may also be configured such that the closer a key point is to other positions of the coil, the higher a scanning priority corresponding to the key point.

In some embodiments, the smaller a first coordinate value of an occluded key point in a first coordinate system, the higher a priority of a scanning protocol corresponding to the key point.

The first coordinate system is an anatomical coordinate system, and is used to describe an anatomical position of a human body. The first coordinate value in the first coordinate system is a coordinate value along a direction from the superior to the inferior of a human body in the first coordinate system, namely, an SI coordinate value.

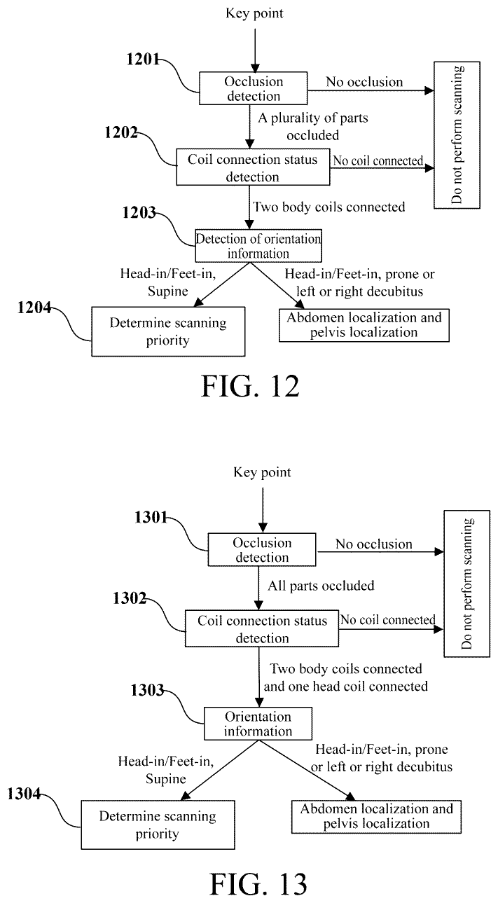

FIG. 12 is still another schematic diagram of a process for determining two or more scanning protocols according to an embodiment of the present application, and FIG. 13 is still another schematic diagram of a process for determining two or more scanning protocols according to an embodiment of the present application.

In the example shown in FIG. 12, the process for determining the scanning protocols includes at step 1201 performing occlusion detection on key points of an examined subject to obtain occlusion information of the key points on the examined subject. When it is detected that a plurality of parts of the examined subject are occluded, for example, the heart, abdomen, pelvis, shoulders, elbows, wrists, hips, knees, and ankles of the examined subject are all occluded, i.e., a plurality of key points corresponding to the above parts are occluded, step 1202 is performed. In addition, if it is detected that no key point on the examined subject is occluded (namely, there is no occlusion) at step 1201, scanning is not performed.

At step 1202, the process includes performing coil connection status detection. When it is detected that a coil is connected to the medical scanning device, step 1203 is performed. The type of coil or which coil is connected to the medical scanning device is determined based on an indication of a first signal. For example, it is determined that two body coils are connected to the medical scanning device based on the indication of the first signal. In addition, if it is detected that no coil is connected to the medical scanning device at step 1202, scanning is not performed.

At step 1203, the process includes detecting orientation information of the examined subject to obtain the orientation information of the examined subject. When the orientation information of the examined subject is a head-in type or a feet-in type, and the examined subject is in a prone or left or right decubitus position, a scanning protocol suitable for abdomen localization and a scanning protocol suitable for pelvis localization, e.g., a 3-plane protocol for abdomen localization and a 3-plane protocol for pelvis localization, are selected. When the orientation information of the examined subject is a head-in type or a feet-in type, and the examined subject is in a supine position, step 1204 is performed.

At step 1204, the process includes determining a scanning priority. The priority of a scanning protocol is determined based on a first coordinate value of a key point in a first coordinate system. The smaller the first coordinate value of the key point, the higher the priority of a scanning protocol corresponding to the key point. For example, when first coordinate values of key points corresponding to the heart, abdomen, pelvis, knees, and ankles that are occluded by coils in the first coordinate system increase sequentially, it is determined that the priorities of the scanning protocols are in the following order: a scanning protocol suitable for heart localization, a scanning protocol suitable for abdomen localization, a scanning protocol suitable for pelvis localization, a scanning protocol suitable for knee localization, and a scanning protocol suitable for ankle localization sequentially, e.g., a 3-plane localizer protocol for heart localization, a 3-plane localizer protocol for abdomen localization, a 3-plane localizer protocol for pelvis localization, a 3-plane localizer protocol for knee localization, and a 3-plane localizer protocol for ankle localization sequentially.

In the example shown in FIG. 13, the process for determining the scanning protocols includes at step 1301 performing occlusion detection on key points of an examined subject to obtain occlusion information of the key points on the examined subject. When it is detected that all anatomical structures of the examined subject are occluded, for example, anatomical structures including, but not limited to, the head, neck, heart, abdomen, pelvis, shoulders, elbows, wrists, hips, and knees, i.e., a plurality of key points corresponding to the above parts are occluded, step 1302 is performed. In addition, if it is detected that no key point on the examined subject is occluded (namely, there is no occlusion) at step 1301, scanning is not performed.

At step 1302, the process includes performing coil connection status detection. When it is detected that a coil is connected to the medical scanning device, step 1303 is performed. The type of coil or which coil is connected to the medical scanning device is determined based on an indication of a first signal. For example, it is determined that two body coils are connected to the medical scanning device and one head coil is connected to the medical scanning device based on the indication of the first signal. In addition, if it is detected that no coil is connected to the medical scanning device at step 1302, scanning is not performed.

At step 1303, the process includes detecting orientation information of the examined subject to obtain the orientation information of the examined subject. When the orientation information of the examined subject is a head-in type or a feet-in type, and the examined subject is in a prone or left or right decubitus position, a scanning protocol suitable for abdomen localization and a scanning protocol suitable for pelvis localization, e.g., a 3-plane localizer protocol for abdomen localization and a 3-plane localizer protocol for pelvis localization, are selected. When the orientation information of the examined subject is a head-in type or a feet-in type, and the examined subject is in a supine position, step 1304 is performed.

At step 1304, the process includes determining a scanning priority. The priority of a scanning protocol is determined based on a first coordinate value of a key point in a first coordinate system. The smaller the first coordinate value of the key point, the higher the priority of the scanning protocol corresponding to the key point. For example, when first coordinate values of key points corresponding to the head, neck, heart, abdomen, pelvis, hips, knees, and ankles that are occluded by coils in the first coordinate system increase sequentially, it is determined that the priorities of the scanning protocols are in the following sequence: a scanning protocol suitable for head localization, a scanning protocol suitable for neck localization, a scanning protocol suitable for heart localization, a scanning protocol suitable for abdomen localization, a scanning protocol suitable for pelvis localization, a scanning protocol suitable for hip localization, a scanning protocol suitable for knee localization, and a scanning protocol suitable for ankle localization, e.g., a 3-plane localizer protocol for head localization, a 3-plane localizer protocol for neck localization, a 3-plane localizer protocol for heart localization, a 3-plane localizer protocol for abdomen localization, a 3-plane localizer protocol for pelvis localization, a 3-plane localizer protocol for hip localization, a 3-plane localizer protocol for knee localization, and a 3-plane localizer protocol for ankle localization sequentially.

In the above example, only some cases in which a scanning protocol is determined based on the location of an occluded key point and the location of the key point relative to a coil respectively are described, but the present application is not limited thereto. When one or more scanning protocols are determined, other cases may also be included. In addition, the priority of a scanning protocol may also be determined based on both the location of a key point and the location of the key point relative to a coil.

The above example illustrates a scenario at step 201 in which a scanning protocol is determined based on three of the following: occlusion information of key points on an examined subject, connection status information between a coil covering the surface of the examined subject and a medical scanning device, and orientation information of the examined subject. For a scenario at step 201 in which a scanning protocol is determined based on two of the following: occlusion information of key points on an examined subject, connection status information between a coil covering the surface of the examined subject and a medical scanning device, and orientation information of the examined subject, reference may be made to the description in the above example.

In some embodiments, at step 202, after the scanning protocol is determined (e.g., the scanning protocol is determined at step 201), the process includes notifying the scanning protocol. For example, the determined scanning protocol is displayed on a user interface (UI). Alternatively, the determined scanning protocol is notified by voice. Alternatively, when two or more scanning protocols are determined, the notification may be performed according to the priorities of the scanning protocols. A scanning protocol with a higher priority may be notified first or the scanning protocol with a higher priority may be displayed at a higher position of a scanning protocol list. Taking FIG. 12 as an example, if the scanning protocols are to be executed in the following sequence: a scanning protocol suitable for heart localization, a scanning protocol suitable for abdomen localization, a scanning protocol suitable for pelvis localization, a scanning protocol suitable for knee localization, and a scanning protocol suitable for ankle localization, then an operator may be notified of each scanning protocol in the above-described sequence.

After the scanning protocols are notified, a user may select a determined scanning protocol, or select to redetermine a scanning protocol, or select other scanning protocols, which may be operated by issuing a voice instruction, pressing a button, or clicking a UI interface. After the user determines a scanning protocol, the medical scanning device may scan an examined subject based on the scanning protocol. The scanning protocol may be a 3-plane localizer protocol. For example, when the scanning protocol is a scanning protocol suitable for head localization, the scanning protocol is a 3-plane localizer protocol for the head, and the medical scanning device scans the head of the examined subject based on the scanning protocol, and recognizes the coronal plane, the sagittal plane, and the horizontal plane of the head based on a scanning result, thereby localizing the head.

The embodiments of the present application further provide an apparatus for determining a scanning protocol in medical imaging, and the contents thereof same as those of the foregoing embodiments are omitted here.

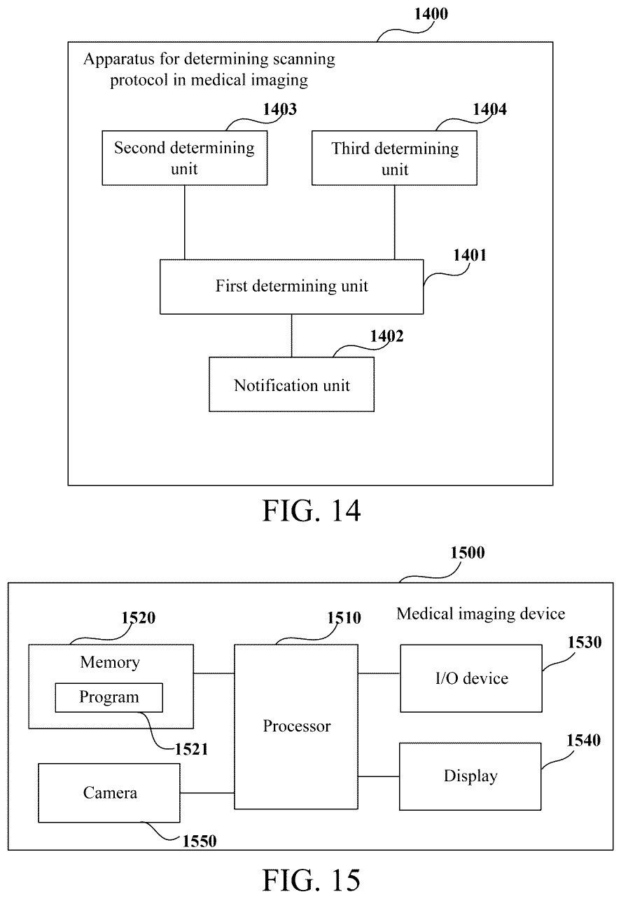

FIG. 14 is a schematic diagram of an apparatus for determining a scanning protocol according to an embodiment of the present application. As shown in FIG. 14, the apparatus 1400 for determining a scanning protocol includes a first determining unit 1401, configured to determine a scanning protocol based on at least two of the following: occlusion information of key points on an examined subject, connection status information between a coil covering the surface of the examined subject and a medical scanning device, and orientation information of the examined subject. The scanning protocol is used by the medical scanning device to scan the examined subject.

The apparatus 1400 also includes a notification unit 1402, configured to notify the scanning protocol. In some embodiments, the apparatus 1400 further includes a second determining unit 1403, which is configured to acquire a two-dimensional image of the examined subject captured by a camera, determine information of key points on the examined subject based on the two-dimensional image, and determine orientation information of the examined subject based on the information of the key points.

In some embodiments, the apparatus 1400 further includes a third determining unit 1404, which is configured to acquire a two-dimensional image and a depth image of the examined subject captured by a camera, determine information of key points on the examined subject based on the two-dimensional image, determine distance information between the surface of the examined subject and the camera based on the depth image, and determine occlusion information of the key points on the examined subject based on the distance information and the information of the key points.

In some embodiments, determining the occlusion information of the key points on the examined subject based on the distance information and the information of the key points includes: when a distance from a region on the surface of the examined subject corresponding to a key point to the camera is less than a first threshold and the area of the region is greater than a second threshold, determining that the key point corresponding to the region is occluded.

In some embodiments, when two or more scanning protocols are determined, the first determining unit 1401 further determines the priorities of the two or more scanning protocols based on at least one of: a positional relationship among a plurality of occluded key points, and a positional relationship of the plurality of occluded key points relative to the coil.

In some embodiments, the smaller a first coordinate value of an occluded key point in a first coordinate system, the higher the priority of the scanning protocol corresponding to the key point; or the closer the occluded key point to the center of the coil, the higher the priority of the scanning protocol corresponding to the key point.

It is worth noting that only the components or modules related to the present application have been described above, but the present application is not limited thereto. The apparatus 1400 for determining a scanning protocol in medical imaging may further include other components or modules, for the specifics of which reference may be made to the related art.

For the sake of simplicity, FIG. 14 only exemplarily illustrates connection relationships or signal directions between various components or modules, but it should be clear to those skilled in the art that various related technologies such as bus connection can be used. The various components or modules described above can be implemented by means of hardware such as a processor or a memory, etc. The embodiments of the present application are not limited thereto.

The above embodiments merely provide illustrative descriptions of the embodiments of the present application. However, the present application is not limited thereto, and suitable variations may be made on the basis of the above embodiments. For example, each of the above embodiments may be used independently, or one or more of the above embodiments may be combined.

Embodiments of the present application further provide a medical imaging system. The medical imaging system includes the apparatus 1400 for determining a scanning protocol in medical imaging as described in the above embodiment, the contents of which are incorporated here. The medical imaging system may, for example, have a computer, a server, a workstation, a laptop computer, a smart phone, or the like. However, the embodiments of the present application are not limited thereto.

FIG. 15 is a schematic diagram of a medical imaging system according to an embodiment of the present application. As shown in FIG. 15, the medical imaging system 1500 may include: one or more processors (for example, central processing units (CPUs)) 1510 and one or more memories 1520. A memory 1520 is coupled to a processor 1510. The memory 1520 may store various types of data. In addition, the memory further stores a program 1521 for information processing, and executes the program 1521 under the control of the processor 1510.

In some embodiments, functions of the apparatus 1400 for determining a scanning protocol in medical imaging are integrated into the processor 1510 for implementation. The processor 1510 is configured to implement the method for determining a scanning protocol in medical imaging as described in the above embodiments of the present application.

In some embodiments, the apparatus 1400 for determining a scanning protocol in medical imaging and the processor 1510 are configured separately. For example, the apparatus 1400 for determining a scanning protocol in medical imaging may be configured as a chip connected to the processor 1510, and functions of the apparatus 1400 for determining a scanning protocol in medical imaging are implemented under the control of the processor 1510.

For example, the processor 1510 is configured to perform the following controls: determining a scanning protocol based on at least two of the following: occlusion information of key points on an examined subject, connection status information between a coil covering the surface of the examined subject and a medical scanning device, and orientation information of the examined subject, the scanning protocol being used by the medical scanning device to scan the examined subject; and notifying the scanning protocol.

In a specific example, the medical imaging system 1500 of FIG. 15 may be the magnetic resonance imaging (MRI) system 100 shown in FIG. 1. The memory 1520 of FIG. 15 may correspond to at least one of the memory 137 and the memory 126 of FIG. 1. For example, the memory 1520 may be independent of at least one of the memory 137 and the memory 126, or the memory 1520 may communicate with at least one of the memory 137 and the memory 126, or the memory 1520 may include at least one of the memory 137 and the memory 126, etc. The processor 1510 of FIG. 15 may correspond to at least one of the CPU 131, the CPU 124, and the image processor 128 of FIG. 1. For example, the processor 1510 may be independent of at least one of the CPU 131, the CPU 124, and the image processor 128, or the processor 1510 may communicate with at least one of the CPU 131, the CPU 124, and the image processor 128, or the processor 1510 may include at least one of the CPU 131, the CPU 124, and the image processor 128, etc.

In addition, as shown in FIG. 15, the medical imaging system 1500 may further include: an input/output (I/O) device 1530, a display 1540, etc. The functions of the foregoing components are similar to those in the prior art. Details are not described herein again.

In addition, as shown in FIG. 15, the medical imaging system 1500 may further include a camera 1550, which captures a subject and generates an image of the subject. The image may be transmitted to the processor 1510, so that the processor 1510 can implement the method for determining a scanning protocol in medical imaging described in the above embodiments of the present application based on the image captured by the camera 1550.

It is worth noting that the medical imaging system 1500 does not necessarily include all of the components shown in FIG. 15. In addition, the medical imaging system 1500 may further include components not shown in FIG. 15, for which reference may be made to the related art.

Embodiments of the present application further provide a computer-readable program that, when executed in a medical imaging system, causes a computer to execute, in the medical imaging system, the method for determining a scanning protocol in medical imaging described in the foregoing embodiments.

The embodiments of the present application further provide a storage medium having a computer-readable program stored therein, wherein the computer-readable program causes a computer to execute, in a medical imaging system, the method for determining a scanning protocol in medical imaging described in any of the foregoing embodiments.

According to the above embodiment, a scanning protocol can be determined based on at least two of the following: occlusion information of key points on an examined subject, connection status information of a coil covering the surface of the examined subject, and orientation information of the examined subject. Therefore, the scanning protocol for the examined subject (for example, the scanning protocol includes a 3-plane localizer protocol) can be automatically determined, and a user need not manually load the scanning protocol outside a scan room, thereby simplifying the scanning process and improving scanning efficiency.

The above apparatus and method of the present application can be implemented by hardware, or can be implemented by hardware in combination with software. The present application relates to such a computer-readable program that when executed by a logic component, the program causes the logic component to implement the foregoing apparatus or a constituent component, or causes the logic component to implement various methods or steps as described above. The present application further relates to a storage medium for storing the above program, such as a hard disk, a disk, an optical disk, a DVD, a flash memory, etc.

The method/apparatus described in view of the embodiments of the present application may be directly embodied as hardware, a software module executed by a processor, or a combination of the two. For example, one or more of the functional block diagrams and/or one or more combinations of the functional block diagrams shown in the drawings may correspond to either respective software modules or respective hardware modules of a computer program flow. The foregoing software modules may respectively correspond to the steps shown in the figures. The foregoing hardware modules can be implemented, for example, by firming the software modules using a field-programmable gate array (FPGA).

The software modules may be located in a RAM, a flash memory, a ROM, an EPROM, an EEPROM, a register, a hard disk, a portable storage disk, a CD-ROM, or any other form of storage medium known in the art. The storage medium may be coupled to a processor, so that the processor can read information from the storage medium and can write information into the storage medium. Alternatively, the storage medium may be a constituent component of the processor. The processor and the storage medium may be located in an ASIC. The software module may be stored in a memory of a mobile terminal, and may also be stored in a memory card that can be inserted into a mobile terminal. For example, if a device (such as a mobile terminal) uses a large-capacity MEGA-SIM card or a large-capacity flash memory apparatus, the software modules can be stored in the MEGA-SIM card or the large-capacity flash memory apparatus.

One or more of the functional blocks and/or one or more combinations of the functional blocks shown in the accompanying drawings may be implemented as a general-purpose processor, a digital signal processor (DSP), an application-specific integrated circuit (ASIC), a field-programmable gate array (FPGA) or other programmable logic devices, discrete gate or transistor logic devices, a discrete hardware assembly, or any appropriate combination thereof for executing the functions described in the present application. The one or more functional blocks and/or the one or more combinations of the functional blocks shown in the accompanying drawings may also be implemented as a combination of computing devices, such as a combination of a DSP and a microprocessor, multiple microprocessors, one or more microprocessors in communication combination with a DSP, or any other such configuration.

The present application is described above with reference to specific implementations. However, it should be clear to those skilled in the art that the foregoing description is merely illustrative and is not intended to limit the scope of protection of the present application. Various variations and modifications may be made by those skilled in the art according to the principle of the present application, and said variations and modifications also fall within the scope of the present application.

1. A method for determining a scanning protocol in medical imaging, comprising:
determining a scanning protocol based on at least two of the following: occlusion information of key points on an examined subject, connection status information between a coil covering the surface of the examined subject and a medical scanning device, and orientation information of the examined subject, the scanning protocol being used by the medical scanning device to scan the examined subject; and
notifying the scanning protocol.

2. The method according to claim 1, further comprising:
acquiring a two-dimensional image of the examined subject captured by a camera;
determining information of key points on the examined subject based on the two-dimensional image; and
determining orientation information of the examined subject based on the information of the key points.

3. The method according to claim 1, further comprising:
acquiring a two-dimensional image and a depth image of the examined subject captured by a camera;
determining information of key points on the examined subject based on the two-dimensional image;
determining distance information between the surface of the examined subject and the camera based on the depth image; and
determining occlusion information of the key points on the examined subject based on the distance information and the information of the key points.

4. The method according to claim 3, wherein
determining the occlusion information of the key points on the examined subject based on the distance information and the information of the key points comprises:
when the distance from a region on the surface of the examined subject corresponding to a key point to the camera is less than a first threshold and the area of the region is greater than a second threshold, determining that the key point corresponding to the region is occluded.

5. The method according to claim 1, further comprising:
when two or more scanning protocols are determined, determining priorities of the two or more scanning protocols based on at least one of: a positional relationship among the plurality of occluded key points, and a positional relationship of the plurality of occluded key points relative to the coil.

6. The method according to claim 5, wherein
the smaller a first coordinate value of an occluded key point in a first coordinate system, the higher the priority of the scanning protocol corresponding to the key point; or
the closer the occluded key point to the center of the coil, the higher the priority of the scanning protocol corresponding to the key point.

7. The method according to claim 1, wherein
upon determining that a key point on the examined subject is occluded and the coil is connected to the medical scanning device,
the scanning protocol is determined based on the orientation information of the examined subject.

8. The method according to claim 1, wherein
upon determining that no key point on the examined subject is occluded, or the coil is not connected to the medical scanning device,
the medical scanning device does not perform scanning.

9. A medical imaging system, comprising a memory and a processor, the memory storing a computer program, and the processor being configured to execute the computer program so as to implement the method for determining a scanning protocol in medical imaging according to claim 1.

10. The medical imaging system according to claim 9, wherein
the medical imaging system comprises a magnetic resonance imaging system, and
the scanning protocol comprises a 3-plane localizer protocol.
